(19) United States
(12) Patent Application Publication
(10) Pub. No.: US 2015/0352537 A1
(43) Pub. Date: Dec. 10, 2015

(54) CRYSTALLINE POROUS SILICOALUMINOPHOSPHATES AND METAL-SUBSTITUTED SILICOALUMINOPHOSPHATES WITH A HIERARCHICAL PORE STRUCTURE COMPRISING MICROPORES AND MESOPORES, METHODS FOR MAKING THE SAME, AND METHODS FOR CONVERTING OXYGENATES TO OLEFINS VIA REACTIONS CATALYZED BY THE SAME

(71) Applicant: UOP LLC, Des Plaines, IL (US)

(72) Inventor: Benjamin Daniel Yuhas, Evanston, IL (US)

(21) Appl. No.: 14/298,749

(22) Filed: Jun. 6, 2014

(51) Int. Cl. B01J 29/85; C01B 39/54; B01J 37/08; B01J 37/04; B01J 37/10; B01J 37/03; C07C 1/20; B01J 29/84

(52) U.S. Cl. CPC B01J 29/85; C07C 1/20; C01B 39/54; B01J 29/84; B01J 37/04; B01J 37/10; B01J 37/031; B01J 37/08; C07C 2529/06

(57) ABSTRACT

Crystalline porous silicoaluminophosphates and metal-substituted silicoaluminophosphates with a hierarchical pore structure comprising micropores and mesopores, methods for making the same, and methods for converting an oxygenate to an olefin via reactions catalyzed by the same are provided. In an embodiment, crystalline porous silicoaluminophosphates with a hierarchical pore structure have mesopores with a minimum dimension of about 25 Å to about 475 Å that constitute at least about 7% of the total pore volume.

### TECHNICAL FIELD

The technical field generally relates to molecular sieve catalysts, methods for making the same, and methods for converting oxygenates to olefins using the same. More particularly, the technical field relates to crystalline silicoaluminophosphates and metal-substituted silicoaluminophosphates with a hierarchical pore structure, methods for making the same, and methods for converting oxygenates to olefins using the same.

### BACKGROUND

Crystalline molecular sieves are among the most important materials in industrial catalysts today. These materials, including zeolites, silicoaluminophosphates (SAPOs), and metal-substituted silicoaluminophosphates (MAPSOs) are typically microporous materials with an average pore dimension on the order of 3-10 Å. However, with a porous network comprising only micropores of this size, catalytic activity in the materials is often limited by mass transfer, potentially limiting production rates.

Therefore, it is desirable to synthesize crystalline molecular sieves with hierarchical pore structure. That is, it is desirable to synthesize crystalline molecular sieves with both the typical micropores as well as larger pores, such as mesopores (which generally have an average pore dimension on the order of about 25 Å to about 475 Å). The presence of the mesopores in the pore structure has the potential to enhance mass transfer capacity of reactants and products into and out of the pore structure, and thus enhance production rates.

Although synthetic pathways are known for preparing mesoporous silicas and zeolitic materials, there are comparatively fewer known synthetic routes for preparing mesoporous silicoaluminophosphates (SAPOs) or metal-substituted silicoaluminophosphates (MAPSOs). Furthermore, most of the known synthetic routes lead to amorphous solids (i.e., solids having no crystalline order) or to solids that are thermally unstable with respect to removal of organic template materials used to form the solids. Additionally, the mesoporous SAPO materials described in the literature do not retain the microporous character that is desired for catalytic applications.

Accordingly, it is desirable to provide novel methods for making SAPO or MAPSO materials with a hierarchical pore structure. Furthermore, other desirable features and characteristics will become apparent from the subsequent detailed description and the appended claims, taken in conjunction with the accompanying drawings and the foregoing technical field and background.

### BRIEF SUMMARY

Crystalline porous silicoaluminophosphates and metal-substituted silicoaluminophosphates with a hierarchical pore structure comprising micropores and mesopores, methods for making the same, and methods for converting methanol to olefins via reactions catalyzed by the same are provided. In one exemplary embodiment, a crystalline porous silicoaluminophosphate with a hierarchical pore structure comprising micropores and mesopores has mesopores with a minimum dimension of about 25 Å to about 475 Å that constitute at least about 7% of a total pore volume.

In another embodiment, a method for making a crystalline porous silicoaluminophosphate or metal-substituted silicoaluminophosphate with a hierarchical pore structure comprising micropores and mesopores is provided. The method comprises admixing a silicon source, an aluminum source, a phosphorous source, optionally a metal source and a plurality of template species with water to form an aqueous gel solution; hydrothermally treating the aqueous gel solution to form and precipitate a solid product with occluded template species; and calcining the precipitated solid product to remove the occluded template species and form the crystalline porous silicoaluminophosphate or metal-substituted silicoaluminophosphate with the hierarchical pore structure comprising micropores and mesopores.

In another embodiment, a method for converting oxygenates to olefins is provided. The method comprises providing a feed stream comprising at least one oxygenate; contacting the feed stream with a crystalline porous silicoaluminophosphate catalyst or metal-substituted silicoaluminophosphate under conditions suitable to catalyze conversion of a oxygenate to a olefins; and forming a product effluent comprising propylene and ethylene such that the product effluent has a propylene to ethylene selectivity ratio of at least about 0.8. In this embodiment, the crystalline porous silicoaluminophosphate or metal-substituted silicoaluminophosphate catalyst has a hierarchical pore structure comprising micropores and mesopores, and the mesopores with a minimum dimension of about 25 Å to about 475 Å constitute at least about 7% of a total pore volume.

### BRIEF DESCRIPTION OF THE DRAWINGS

FIG. 1 is an SEM image of an exemplary crystalline porous SAPO material with hierarchical pore structure according to an embodiment described herein.

FIG. 2 is a schematic diagram of an apparatus and method for converting methanol to olefins via reaction catalyzed by an exemplary crystalline porous SAPO material with hierarchical pore structure as described herein.

FIG. 3 is a graph showing pore size distributions of exemplary crystalline porous SAPO and MAPSO materials with hierarchical pore structure according exemplary embodiments prepared using a variety of polyprotic organic acids as one of a plurality of template species. Details are discussed in Example 1.

### DETAILED DESCRIPTION

The following detailed description is merely exemplary in nature and is not intended to limit the exemplary methods or apparatuses described herein. Furthermore, there is no intention to be bound by any theory presented in the preceding background or the following detailed description.

Crystalline porous silicoaluminophosphate (SAPO) and metal-substituted silicoaluminophosphates (MAPSO) with a hierarchical pore structure comprising micropores and mesopores are described herein. SAPOs with a porous structure comprising pore volumes in both the mesopore and micropore ranges described herein show increased catalytic activity, particularly with respect to methanol to olefin (MTO) catalysis, relative to SAPOs with reduced mesopore volume. Further, when crystalline porous silicoaluminophosphate (SAPO) or metal-substituted silicoaluminophosphate (MAPSO) material with porosity in the mesoporous range described herein have the chabazite (CHA) crystal structure, a propylene/ethylene ratio in the products of the catalyzed reactions is higher than that generated with a SAPO or MAPSO with porosity in the microporous range alone.

As used herein, micropores are pores with a minimum internal dimension (i.e., pore size) of about 25 Å or less. Mesopores are pores with a minimum internal dimension (i.e., pore size) of about 25 Å to about 475 Å. A porous material is said to have a “hierarchical” pore structure when the material's pore size distribution includes both micropores and mesopores.

Methods described herein provide a synthesis route for crystalline, porous SAPOs or MAPSOs with a hierarchical pore structure. In some embodiments, crystalline porous SAPOs or MAPSOs prepared according to methods described herein have a hierarchical pore structure such that at least about 7%, such as at least about 10%, such as at least about 15%, of the pore volume of the material is from mesopores. In some embodiments, the mesopores contributing to this volume have a minimum dimension of about 25 Å to about 475 Å. In some embodiments, crystalline porous SAPOs or MAPSOs prepared according to methods described herein have a peak in their pore volume distribution (i.e., a peak pore volume) within the range of about 90 Å to about 200 Å.

In some embodiments, synthesis is conducted via a single batch process utilizing a plurality of template species. For example, in some methods a quaternary ammonium species and an ionized polyprotic organic acid may each be used in the same batch synthesis as separate template species. Use of a plurality of template species in tandem allows for the formation of a hierarchical pore structure in a crystalline product in a single step.

In an exemplary embodiment, a synthesis method follows standard sol-gel techniques but employs a plurality of template species as indicated above in a single batch process. That is, an aqueous gel solution comprising a silicon source, an aluminum source, a phosphorous source, and a plurality of template species is prepared by mixing each of these constituents in water to form a gel. In some embodiments, the aqueous gel solution is prepared such that the gel molar oxide ratio (GMOR) meets the following general conditions: 0.9-1.1 phosphorous source (as P₂O₅):1 aluminum source (as Al₂O₃):0.05-0.15 silicon source (as SiO₂):40-60 water, plus each of the plurality of the template species. In some embodiments, two template species, such as a quaternary amine and a polyprotic organic acid, are used. In some particular embodiments, the aqueous gel solution is prepared such that the GMOR meets the following general conditions: 0.9-1.1 phosphorous source (as P₂O₅):1 aluminum source (as Al₂O₃):0.05-0.15 silicon source (as SiO₂):2.5-3.0 quaternary amine:0.25-0.4 polyprotic organic acid:40-60 water. In some related embodiments, the molar ratio of quaternary amine:polyprotic organic acid is within the range of about 7.5-10.0:1. In some embodiments, the resulting crystalline porous SAPO material has a silicon content of about 3.0 mol % to about 10.0 mol %, such as about 4.0 mol % to about 10.0 mol %, such as about 5.0 mol % to about 10.0 mol %. In some embodiments, the silicon content is about 3 mol % to about 8.0 mol %, such as about 4.0 mol % to about 8.0 mol %, such as about 5.0 mol % to about 8.0 mol %.

Selection of an appropriate silicon source is within the purview of one of skill in the art. One non-limiting example of a typically silicon source is colloidal silica sol. Similarly, selection of an appropriate aluminum source is within the purview of one of skill in the art. One non-limiting example of a typical aluminum source is pseudo-boehmite Finally, selection of an appropriate phosphorous source is within the purview of one of skill in the art. A non-limiting example of a typical phosphorous source is orthophosphoric acid.

In some embodiments, one of the plurality of template species is a quaternary ammonium species. As used herein, quaternary ammonium species are positively charged polyatomic ions of the structure NR₄⁺ with R being alkyl or aryl groups. Numerous quaternary ammonium species are known in the art and may be used as a template species in the methods herein. For instance, in some embodiments, the quaternary ammonium template species may be defined by the formula: (R₁R₂R₃R₄(N+))(OH—), where R₁, R₂, R₃, and R₄ are independently methyl, ethyl, propyl, or butyl. In some particular embodiments, the quaternary ammonium template species is tetraethylammonium hydroxide or tetramethylammonium hydroxide.

In some embodiments, one of the template species is a polyprotic organic acid. As used herein a polyprotic organic acid is defined as an acid having two or more ionizable protons bound to separate carboxylate moieties. Polyprotic organic acids particularly useful as template species in methods described herein satisfy at least one of two conditions, namely: A) the polyprotic organic acid has a minimum water solubility of 3.0 g acid per 100 ml water at ambient temperature and pressure; and B) the polyprotic organic acid has a melting temperature of about 100° C. to about 200° C. at atmospheric pressure. Non-limiting polyprotic organic acids satisfying at least one of these conditions include: citric acid, nitrilotriacetic acid, tricarballylic acid, citraconic acid, malic acid, suberic acid, sebacic acid, tartaric acid, malonic acid, dodecanedioic acid, tetradecanedioic acid, adipic acid, and glutaric acid. In some embodiments, a template species is a polyprotic organic acid selected from the group consisting of citric acid, tricarballylic acid, citraconic acid, and tartaric acid.

In some embodiments, a non-silicon, non-aluminum metal is added into the synthesis mixture to prepare MAPSO product. The choice of metal may be any element in groups 2 through 13 of the periodic table, with preferred metals being Mg, Ca, Sc, Ti, V, Cr, Mn, Fe, Co, Ni, Cu, Zn, Ga, and most preferred metals being Co or Cu. The source of this metal is a salt, such as a halide, nitrate, sulfate, carbonate, or acetate, and is most advantageously an acetate.

Once the aqueous gel solution comprising the phosphorous source, the aluminum source, the silicon source, the additional metal if present, and the plurality of template species is prepared, the aqueous gel solution is hydrothermally treated with stirring to form and precipitate a SAPO or MAPSO solid product. In an exemplary embodiment, the aqueous gel solution may be subjected to a temperature of about 150° C. to about 200° C., or such as about 170° C. to about 185° C., or such as about a temperature of about 175° C., for at least a sufficient amount of time for formation and precipitation of a solid product to occur. The minimum amount of time for formation and precipitation to occur varies with the specific composition of the aqueous gel solution and the temperature of the hydrothermal treatment, and hydrothermally treating the gel for an excess amount of time is typically not harmful. In some embodiments the aqueous gel solution may be hydrothermally treated for a period of about 50 hours to about 70 hours. In an exemplary embodiment, the hydrothermal reaction is conducted at about 175° C. with stirring for at least about 60 hours.

After formation and precipitation, solidified product is isolated from the solution by any suitable method, e.g., filtering, centrifugation, etc., washed with water, and dried. Drying may be conducted under a variety of conditions, including at ambient temperatures (e.g., about 75° C.) or at temperatures above typical ambient conditions, such as to a temperature of about 90° C. to about 110° C., in air until the solidified product is dry. In an exemplary embodiment, the solidified product is isolated by centrifugation, washed with water, and dried overnight at about 100° C. in air.

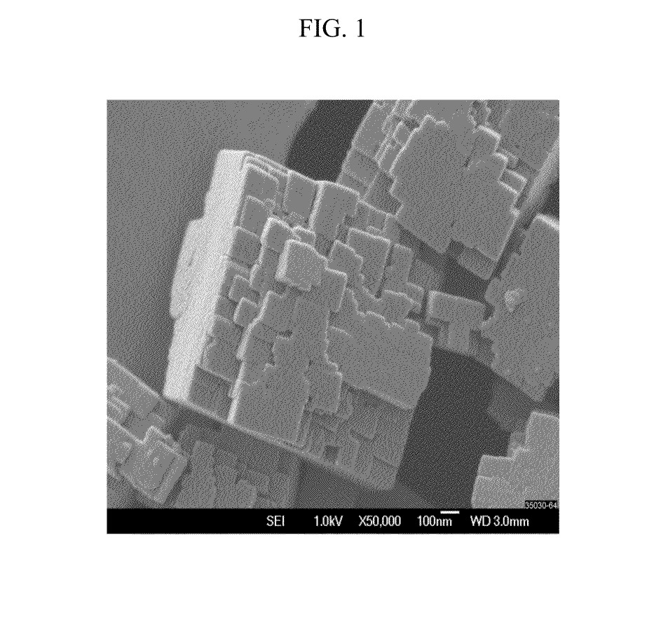

Dried solidified products are then calcined in air at a temperature sufficiently high to remove the occluded template species. In some embodiments, the solidified products are calcined in air at a temperature of up to about 650° C., such as about 500° C. to about 650° C. After removal of the occluded template species, the resulting crystalline porous SAPO or MAPSO is a single phase material with the desired hierarchical pore structure. An SEM image of an exemplary crystalline porous SAPO prepared according to methods described herein is shown in FIG. 1, where it is seen that the exemplary crystalline porous SAPO is a single phase material.

As provided herein, the surface area and pore volume distribution of a particular porous material may be determined, for example, by nitrogen adsorption using the conventional BET method of analysis (see, e.g., J. Am. Chem. Soc. 1938, 60, 309-16) as implemented in Micrometrics ASAP 2010 software (from Micrometrics Instrument Corporation). The pore volume and pore size distribution of a particular porous material may be determined from the Barrett-Joyner-Halenda (BJH) analysis of the nitrogen adsorption isotherm as implemented in Micrometrics ASAP 2010 software (from Micrometrics Instrument Corporation), and is expressed as the differential adsorption of nitrogen versus the pore dimension. As will be appreciated by those of skill in the art, the BJH analysis is generally suitable to determine the pore size distribution for pores having a dimension of about 10 Å to about 1000 Å. The pore fraction of a particular range is then determined by summing the pore volume in the range of interest and dividing by the total pore volume. For instance, the % pore volume in the mesopore range in the materials described herein is determined by determining the pore volume of pores with dimensions within the range of about 25 Å to about 475 Å and dividing by the total pore volume (that is, the volume of pores with dimensions within the range of about 9.5 Å to about 1250 Å).

As described above, crystalline porous SAPOs or MAPSOs prepared according to methods described herein have a hierarchical pore structure comprising micropores and mesopores. Exemplary crystalline porous SAPO or MAPSO materials prepared according to the methods provided herein may be made with a volume fraction of pores in the mesopore range of at least about 7% to about 50%, such as about 10% to about 50%, such as about 15% to about 50%. In some embodiments, crystalline porous SAPOs or MAPSOs prepared according to methods described herein have a peak pore volume within the range of about 90 Å to about 200 Å.

The hierarchical pore structure of the crystalline porous SAPOs described herein has a significant impact on the catalytic behavior of the crystalline porous SAPOs, particularly when employed as catalysts to convert oxygenates to olefins. As will be appreciated, the term oxygenate is used to describe organic compounds with oxygen in their chemical structure. Exemplary oxygenates include alcohols and ethers. The term olefin is used to describe unsaturated hydrocarbons with one carbon-carbon double bond.

Thus, in another aspect, methods of converting an oxygenate to an olefin via a catalyzed reaction are provided. In these methods, a feed stream comprising an oxygenate such as methanol or dimethyl ether is contacted with a catalyst comprising at least one crystalline porous SAPO with a hierarchical pore structure as described herein under conditions suitable for conversion of an oxygenate to an olefin. In some embodiments, the oxygenate is methanol and/or dimethyl ether, and the olefin is ethylene and/or propylene. Conditions employed to use the catalysts in a oxygenate to olefin conversion reaction can be readily identified by a person of ordinary skill in the art. In an exemplary embodiment, a crystalline porous SAPO with a hierarchical pore structure as described herein may be sized by a 40/60 standard mesh, and a portion of the sized material placed in a fixed bed reactor. In this exemplary embodiment, the reactor is heated to a temperature of about 450° C. and a methanol and/or dimethyl ether-comprising feed is introduced to the reactor at a pressure of about 5 psig. A reactor effluent comprising light olefins including ethylene and propylene is generated via catalytic conversion.

In SAPO catalyzed methanol to olefin reactions, generation of ethylene is typically favored over generation of propylene. However, increased propylene production is typically desired as propylene is a higher value product. It has surprisingly been discovered that methanol to olefin reactions catalyzed by the crystalline porous SAPOs described herein exhibit a higher selectivity towards propylene formation than is seen with methanol to olefin reactions catalyzed by prior art SAPOs. For instance, reactions catalyzed by crystalline porous SAPOs with a hierarchical pore structure as described herein exhibit propylene selectivity such that a propylene to ethylene selectivity ratio is greater than 0.7, with increasing propylene to ethylene ratios evident with increasing mesopore volume fraction. In some embodiments, reactions catalyzed by crystalline porous SAPOs with a hierarchical pore structure as described herein have a propylene to ethylene selectivity ratio greater than or equal to about 0.8, such as greater than or equal to 0.85. In some embodiments, the propylene to ethylene selectivity ratio is from about 0.8 to about 0.9, such as from about 0.85 to about 0.9.

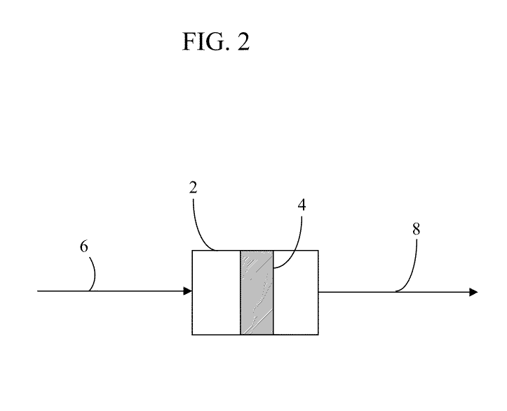

Also described herein are systems for converting an oxygenate to an olefin in a SAPO catalyzed reaction. The following exemplary systems are described with reference to FIG. 2. Exemplary systems include a reaction vessel 2 configured to contain one or more catalytically active crystalline porous SAPOs with a hierarchical pore structure 4. The reaction vessel 2 is configured to receive a oxygenate-containing feed stream 6, and contact the feed stream 6 with the catalytically active crystalline porous SAPO with the hierarchical pore structure 4 under reaction conditions effective to convert an oxygenate to an olefin and produce an olefin-containing effluent 8. In some embodiments, the feed stream 6 comprises one or more of methanol and dimethyl ether. In these embodiments, the olefin-containing effluent 8 comprises one or more of propylene and ethylene. In some embodiments, systems and methods utilizing at least one crystalline porous SAPO with the hierarchical pore structure 4 result in an effluent 8 that has an increased proportion of propylene relative ethylene as compared to similar effluents resulting from prior art crystalline microporous SAPO catalysts. In some embodiments, the propylene to ethylene ratio in the effluent is greater than or equal to about 0.8, such as greater than or equal to 0.85. In some embodiments, the propylene to ethylene selectivity ratio is about 0.8 to about 0.9, such as about 0.85 to about 0.9.

Note that reference to the specific arrangement in FIG. 2 is not meant to limit the apparatus and method to the details disclosed therein. Furthermore, FIG. 2 is a schematic illustration and does not show a number of details for the process arrangement such as pumps, compressors, valves, and recycle lines that are well-known to those skilled in the art.

Those having skill in the art, with the knowledge gained from the present disclosure, will recognize that various changes could be made in the methods described herein without departing from the scope of the present invention. Mechanisms used to explain theoretical or observed phenomena or results, shall be interpreted as illustrative only and not limiting in any way the scope of the appended claims.

The following examples include representative methods of making crystalline porous SAPOs or MAPSOs with a hierarchical pore structure using various polyprotic organic acids as one of a plurality of template species. These examples are not to be construed as limiting as other equivalent embodiments will be apparent in view of the present disclosure and appended claims.

### EXAMPLES

Several exemplary crystalline porous SAPO or MAPSO materials with hierarchical pore structure were prepared using a variety of polyprotic organic acids in aqueous gel solutions with different GMORs. Each synthesis reaction was conducted according to the methods described above. The pore structures of the resulting crystalline porous SAPO materials were characterized; the crystalline structure and silicon content of the materials were determined, and the materials were tested for performance as methanol to olefin catalysts. The details of synthesizing, characterizing, and testing five exemplary crystalline porous SAPO materials are provided below.

A first exemplary crystalline porous SAPO material was prepared by adding 12.82 g of citric acid (99%, Aldrich) to 19.28 g of distilled water (H₂O) and stirring until a clear solution was obtained. This solution was then added to 278.06 g of a 35% tetraethylammonium hydroxide (TEAOH) solution (Sachem, Inc.) and mixed. 5.95 g of colloidal silica (SiO₂) (Ludox, AS40, 40%, Aldrich) were added, followed by 60.94 g of orthophosphoric acid (H₃PO₄) (85%, Fisher Scientific) and 38.02 g of pseudo-boehmite alumina (Al₂O₃) (Versal 251, UOP). The mixture was then stirred to form a gel. The resulting aqueous gel solution had the following gel molar oxide ratio (GMOR) (with phosphorous expressed as equivalents of phosphorous pentoxide (P₂O₅)):

1.0 P₂O₅:1.0 Al₂O₃:0.15 SiO₂:2.5 TEAOH:0.25 Citric Acid:50 H₂O

The aqueous gel solution was stirred for 2 hours and transferred to a stainless steel autoclave. In the autoclave, the aqueous gel solution gel was heated at 175° C. with stirring for 66 hours, then allowed to cool to room temperature. Solid products were isolated by centrifugation, washed with distilled water, and dried overnight at 100° C. in air. X-ray diffraction of the as-synthesized solids showed pure SAPO-34 (chabazite phase). The silicon content of the solids was determined by inductively coupled plasma (ICP) spectroscopy as 8.0 mol %.

A second exemplary crystalline porous SAPO material was prepared by adding 7.12 g of distilled water (H₂O) to 319.30 g of a 35% tetraethylammonium hydroxide (TEAOH) solution (Sachem, Inc.) and stirring. 64.15 g of orthophosphoric acid (H₃PO₄) (85%, Fisher Scientific) was added to this solution, followed by 35.82 g of pseudo-boehmite alumina (Al₂O₃) (Catapal B, Sasol), 1.90 g of colloidal silica (SiO₂) (Ludox, AS40, 40%, Aldrich), and 19.65 g of citric acid (99%, Aldrich). The mixture was then stirred to form a gel. The resulting aqueous gel solution had the following gel molar oxide ratio (GMOR) (with phosphorous expressed as equivalents of phosphorous pentoxide (P₂O₅)):

1.1 P₂O₅:1.0 Al₂O₃:0.05 SiO₂:3.0 TEAOH:0.4 Citric Acid:55 H₂O

The aqueous gel solution was stirred for 30 minutes and transferred to a stainless steel autoclave. In the autoclave, the aqueous gel solution was heated at 175° C. with stirring for 62 hours, then allowed to cool to room temperature. Solid products were isolated by centrifugation, washed with distilled water, and dried overnight at 100° C. in air. X-ray diffraction of the as-synthesized solids showed pure SAPO-34 (chabazite phase). The silicon content of the solids was determined by inductively coupled plasma (ICP) spectroscopy as 5.9 mol %.

A third exemplary crystalline porous SAPO material was prepared by adding 8.37 g of distilled water (H₂O) to 120.40 g of a 35% tetraethylammonium hydroxide (TEAOH) solution (Sachem, Inc.) and stirring. 26.40 g of orthophosphoric acid (H₃PO₄) (85%, Fisher Scientific) was added to this solution, followed by 16.44 g of pseudo-boehmite alumina (Al₂O₃) (Versal 250C, UOP), 2.58 g of colloidal silica (SiO₂) (Ludox, AS40, 40%, Aldrich), and 5.10 g of tricarballylic acid (99%, Aldrich). The mixture was then stirred to form a gel. The resulting aqueous gel solution had the following gel molar oxide ratio (GMOR) (with phosphorous expressed as equivalents of phosphorous pentoxide (P₂O₅)):

1.0 P₂O₅:1.0 Al₂O₃:0.15 SiO₂:2.5 TEAOH:0.25 Tricarballylic Acid:50 H₂O

The aqueous gel solution was stirred for 30 minutes and transferred to a stainless steel autoclave. In the autoclave, the aqueous gel solution was heated at 175° C. with stirring for 62 hours, then allowed to cool to room temperature. Solid products were isolated by centrifugation, washed with distilled water, and dried overnight at 100° C. in air. X-ray diffraction of the as-synthesized solids showed pure SAPO-34 (chabazite phase). The silicon content of the solids was determined by inductively coupled plasma (ICP) spectroscopy as 6.0 mol %.

A fourth exemplary crystalline porous SAPO material was prepared by adding 21.14 g of distilled water (H₂O) to 303.41 g of a 35% tetraethylammonium hydroxide (TEAOH) solution (Sachem, Inc.) and stirring. 66.53 g of orthophosphoric acid (H₃PO₄) (85%, Fisher Scientific) was added to this solution, followed by 41.43 g of pseudo-boehmite alumina (Al₂O₃) (Versal 250C, UOP), 6.50 g of colloidal silica (SiO₂) (Ludox, AS40, 40%, Aldrich), and 9.48 g of citraconic acid (99%, Aldrich). The mixture was then stirred to form a gel. The resulting aqueous gel solution had the following gel molar oxide ratio (GMOR) (with phosphorous expressed as equivalents of phosphorous pentoxide (P₂O₅):

1.0 P₂O₅:1.0 Al₂O₃:0.15 SiO₂:2.5 TEAOH:0.25 Citraconic Acid:50 H₂O

The aqueous gel solution was stirred for 30 minutes and transferred to a stainless steel autoclave. In the autoclave, the aqueous gel solution was heated at 175° C. with stirring for 59 hours, then allowed to cool to room temperature. Solid products were isolated by centrifugation, washed with distilled water, and dried overnight at 100° C. in air. X-ray diffraction of the as-synthesized solids showed pure SAPO-34 (chabazite phase). The silicon content of the solids was determined by inductively coupled plasma (ICP) spectroscopy as 6.8 molar percent.

A fifth exemplary crystalline porous MAPSO material was prepared by adding 13.33 g of citric acid (99%, Aldrich) to 19.35 g of distilled water (H₂O) and stirring until a clear solution was obtained. This solution was then added to 289.45 g of a 35% tetraethylammonium hydroxide (TEAOH) solution (Sachem, Inc.) and mixed. 6.19 g of colloidal silica (SiO₂) (Ludox, AS40, 40%, Aldrich) were added, followed by 63.43 g of orthophosphoric acid (H₃PO₄) (85%, Fisher Scientific) and 37.81 g of pseudo-boehmite alumina (Al₂O₃) (Catapal B, Sasol). Finally, 10.48 g of cobalt (II) acetate (Aldrich) was added. The mixture was then stirred to form a gel. The resulting aqueous gel solution had the following gel molar oxide ratio (GMOR) (with phosphorous expressed as equivalents of phosphorous pentoxide (P₂O₅)):

1.0 P₂O₅:1.0 Al₂O₃:0.15 SiO₂:0.15 Co(OAc)₂:2.5 TEAOH:0.25 Citric Acid:50 H₂O

The aqueous gel solution was stirred for 30 minutes and transferred to a stainless steel autoclave. In the autoclave, the aqueous gel solution was heated at 175° C. with stirring for 64 hours, then allowed to cool to room temperature. Solid products were isolated by centrifugation, washed with distilled water, and dried overnight at 100° C. in air. X-ray diffraction of the as-synthesized solids showed pure SAPO-34 (chabazite phase).

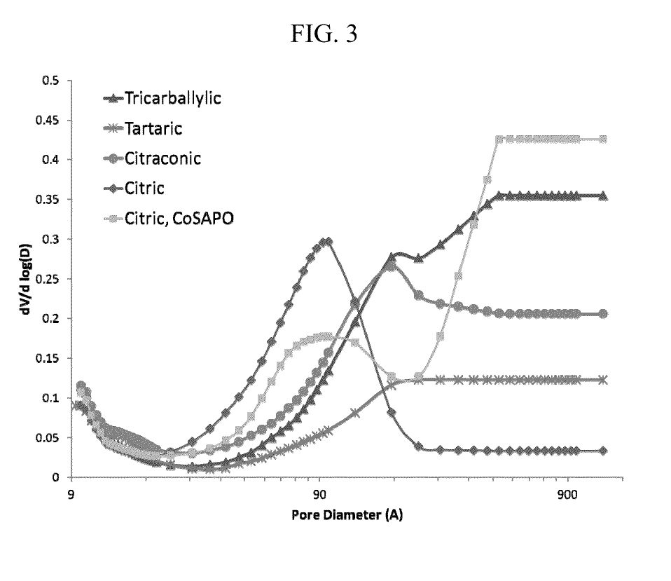

As-synthesized solids from five exemplary reactions (using citric, tricarballylic, tartaric, citraconic, and citric acids as one of the plurality of template species, respectively) were calcined in air at about 600° C. for 4 hours, ramping up the temperature from room temperature to the calcining temperature at a rate of about 2° C. per minute. After calcining, the pore volume distributions of the solids were determined. Plots of the pore volume distributions are shown in FIG. 3. Each exemplary SAPO or MAPSO material was determined to have greater than about 7% mesopore volume, and as seen in FIG. 3, each exemplary SAPO or MAPSO material had a peak pore volume within the range of about 90 Å to about 200 Å.

Finally, a portion of several calcined solids prepared as described above were sized by a 40/60 standard stainless steel mesh. 325 mg of each sized solid were placed in a fixed bed reactor for methanol to olefin catalysis testing. The bed was heated to about 450° C., and a methanol feed stream was introduced to the reactor at a pressure of approximately 5 psig. The reaction products were monitored by gas chromatography and each catalyst exhibited enhanced selectivity for generation of propylene as compared to SAPO catalysts without mesoporosity.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A composition of matter comprising a crystalline porous silicoaluminophosphate or metal-substituted silicoaluminophosphate with a hierarchical pore structure comprising micropores and mesopores, wherein mesopores with a minimum dimension of about 25 Å to about 475 Å constitute at least about 7% of a total pore volume.

2. The composition of claim 1, wherein mesopores with a minimum dimension of about 25 Å to about 475 Å constitute about 7% to about 50% of the total pore volume.

3. The composition of claim 1, wherein mesopores with a minimum dimension of about 25 Å to about 475 Å constitute about 10% to about 50% of the total pore volume.

4. The composition of claim 1, wherein mesopores with a minimum dimension of about 25 Å to about 475 Å constitute about 15% to about 50% of the total pore volume.

5. The composition of claim 1, wherein the hierarchical pore structure has a pore volume distribution such that there is a peak pore volume of mesopores with a minimum dimension of about 90 Å to about 200 Å.

6. The composition of claim 1, wherein the crystalline porous silicoaluminophosphate comprises about 3.0% to about 10.0 mol % silicon.

7. The composition of claim 1, wherein the crystalline porous silicoaluminophosphate comprises about 3.0% to about 8.0 mol % silicon.

8. A method for making a crystalline porous silicoaluminophosphate or metal-substituted silicoaluminophosphate with a hierarchical pore structure comprising micropores and mesopores, the method comprising:
admixing a silicon source, an aluminum source, a phosphorous source, optionally a metal source, and a plurality of template species with water to form an aqueous gel solution;
hydrothermally treating the aqueous gel solution to form and precipitate a solid product with occluded template species; and
calcining the precipitated solid product to remove the occluded template species and form a crystalline porous silicoaluminophosphate with a hierarchical pore structure comprising micropores and mesopores.

9. The method of claim 8, wherein mesopores with a minimum dimension of about 25 Å to about 475 Å constitute about 7% to about 50% of the total pore volume.

10. The method of claim 8, wherein mesopores with a minimum dimension of about 25 Å to about 475 Å constitute about 10% to about 50% of the total pore volume.

11. The method of claim 8, wherein mesopores with a minimum dimension of about 25 Å to about 475 Å constitute about 15% to about 50% of the total pore volume.

12. The method of claim 8, wherein the hierarchical pore structure has a pore volume distribution such that there is a peak pore volume of mesopores with a minimum dimension of about 90 Å to about 200 Å.

13. The method of claim 8, wherein the aqueous gel solution comprises amounts of the silicon source, the aluminum source, and the phosphorous source such that the aqueous gel solution has a gel molar oxide ratio of 0.9-1.1 phosphorous as phosphorous pentoxide (P₂O₅):1 aluminum as alumina (Al₂O₃):0.05-0.15 silicon as silica (SiO₂):40-60 water.

14. The method of claim 8, wherein the plurality of template species comprises a quaternary amine.

15. The method of claim 14, wherein the quaternary amine is defined by the formula:
(R₁R₂R₃R₄(N+))(OH—),
where R₁, R₂, R₃, and R₄ are independently methyl, ethyl, propyl, or butyl.

16. The method of claim 8, wherein the plurality of template species comprises tetraethylammonium hydroxide or tetramethylammonium hydroxide.

17. The method of claim 8, wherein the plurality of template species comprises a polyprotic organic acid.

18. The method of claim 17, wherein the polyprotic organic acid is selected from the group consisting of citric acid, nitrilotriacetic acid, tricarballylic acid, citraconic acid, malic acid, suberic acid, sebacic acid, tartaric acid, malonic acid, dodecanedioic acid, tetradecanedioic acid, adipic acid, and glutaric acid.

19. The method of claim 8, wherein the aqueous gel solution comprises amounts of the silicon source, the aluminum source, the phosphorous source, and the plurality of template species including a quaternary amine and a polyprotic organic acid such that the aqueous gel solution has a gel molar oxide ratio of 0.9-1.1 phosphorous as phosphorous pentoxide (P₂O₅):1 aluminum as alumina (Al₂O₃):0.05-0.15 silicon as silica (SiO₂):2.5-3.0 quaternary amine:0.25-0.4 polyprotic organic acid:40-60 water.

20. A method for converting an oxygenate to an olefin comprising:
providing a feed stream comprising an oxygenate;
contacting the feed stream with a crystalline porous silicoaluminophosphate catalyst under conditions suitable to catalyze conversion of an oxygenate to an olefin, wherein the crystalline porous silicoaluminophosphate catalyst has a hierarchical pore structure comprising micropores and mesopores, and wherein mesopores with a minimum dimension of about 25 Å to about 475 Å constitute at least about 7% of a total pore volume; and
forming a product effluent comprising propylene and ethylene such that the product effluent has a propylene to ethylene selectivity ratio of at least about 0.8.
